(19) United States
(12) Patent Application Publication
(10) Pub. No.: US 2026/0048012 A1
(43) Pub. Date: Feb. 19, 2026

(54) METHOD FOR THE PRODUCTION OF PLANT-DERIVED NANOVESICLES (PDVs) AND THEIR APPLICATIONS

(71) Applicant: UNIVERSITÀ DEGLI STUDI DI MILANO, Milano (IT)

(72) Inventors: Carmen LAMMI, Milano (IT); Davide Lecca, Milano (IT); Umberto Musazzi, Nerviano (IT); Maria Pia Abbracchio, Milano (IT); Anna Arnoldi, Milano (IT); Luigi Sironi, Nova Milanese (IT); Davide Marangon, Vercelli (IT)

(21) Appl. No.: 19/371,571

(22) Filed: Oct. 28, 2025

Related U.S. Application Data
(63) Continuation-in-part of application No. PCT/EP2024/061065, filed on Apr. 23, 2024.

(30) Foreign Application Priority Data
Apr. 28, 2023 (WO) 102023000008343

(51) Int. Cl. A61K 9/1277

(52) U.S. Cl. CPC A61K 9/1277

(57) ABSTRACT

The present invention concerns a method for the production, purification, and stabilization of plant-derived nanovesicles (PDVs). It also concerns a pharmaceutical composition comprising the PDVs obtained by this method for hypocholesterolemic, hypoglycemic, hypolipidemic, anti-ageing, and antioxidant use.

The present invention concerns a method for the production, purification, and stabilization of plant-derived nanovesicles (PDVs).

It also concerns a composition containing the plant-derived nanovesicles obtained by this method for hypocholesterolemic, hypoglycemic, hypolipidemic, antiageing, and antioxidant use.

All eukaryotic and prokaryotic organisms produce vesicles under different physiological or pathological conditions. They are released from inside the cell and are produced using the cell membrane itself which incorporates the load to be transported and “buds” towards the outside.

Plant-derived vesicles have peculiar structures and compositions, which differ from those of animals. Plants produce various types of nano- and microvesicles located in the extracellular space or in the cytoplasm. In general, plant-derived vesicles are round-shaped structures in which proteins, lipids, nucleic acids (RNA, miRNA, DNA) and secondary metabolites are wrapped in a lipid bilayer structure; in plants, these vesicles are crucial in the intercellular transport of molecules involved in defense mechanisms against pathogens, in the response to biotic or abiotic stresses and in long-distance gene regulation.

However, a relevant gap exists regarding their functional roles and biological implications in human health. In fact, few preliminary studies have highlighted that plant-derived vesicles exert intestinal anti-inflammatory and antioxidant activities in some cellular and animal models.

Furthermore, some evidence indicates that these vesicles are resistant to digestive enzymes and reach the intestine intact where they are selectively absorbed by intestinal macrophages (Molecular Therapy, 2014, 22(3), 522-534).

Recently, it has been shown that extracellular nanovesicles from orange juice may be useful for the treatment of intestinal complications associated with obesity. Lemon extracellular vesicles exert antitumor activity through increased expression of apoptotic factors and reduce pro-angiogenic factors in serum (J. Proteomics, 2018, 173, 1-11).

Despite these indications regarding their potential biological activity, plant-derived vesicles are still poorly studied, probably due to the absence of a single standardized and efficient protocol for extracting them from different plant matrices.

The most applied approach is based on the ultracentrifugation technique which however presents some drawbacks, such as low recovery rates, low yield, as well as a significant waste of time. Furthermore, the use of this technique leads to the formation of numerous aggregated vesicles, thus influencing their functionality and biological activity.

The Applicant has now surprisingly identified a method for the mechanical plant tissue processing production, purification, and stabilization of plant-derived nanovesicles (PDVs), which overcomes the drawbacks of the prior art.

Furthermore, the aforementioned process is industrially scalable and allows the PDVs thus obtained to be preserved and stabilized in the long term.

### BRIEF DESCRIPTION OF THE DRAWINGS

FIG. 1 shows viability of human hepatic HepG2 cells tested by MTT assay after treatment with rocket-derived nanovesicles 0.5 and 1 mg/ml for 24 hours.

FIG. 2 shows TRANSWELL® membrane system

FIGS. 3A and 3B show Fluorescence of the apical and basolateral solutions after incubation of the rocket-derived nanovesicles for 2 and 4 hours with human intestinal Caco-2 cells.

FIGS. 4A, 4B, and 4C show modulation of the low-density lipoprotein receptor (LDLR) pathway by rocket-derived nanovesicles.

FIGS. 5A and 5B show regulation of the AMPK pathway.

FIG. 6 shows fluorescent assay of LDL uptake after treatments of human hepatic HepG2 cells with rocket-derived nanovesicles.

FIG. 7 shows modulation of the low-density lipoprotein receptor (LDLR) pathway by rocket-derived nanovesicles and rocket-derived lysed vesicles.

FIGS. 8A and 8B show the effects of rocket-derived nanovesicles on the PCSK9 pathway.

FIGS. 9A and 9B show the effects of rocket-derived nanovesicles on the levels of FASN and PPARγ proteins.

FIGS. 10A and 10B show the effects of rocket-derived nanovesicles on the Akt pathway and glucose transporter 4 (GLUT4).

FIG. 11 shows secondary metabolites present in the rocket-derived nanovesicles, identified with HR-HPLC-MS.

FIG. 12 shows nanoparticle tracking analysis (NTA) graphs of rosemary-derived nanovesicles (RVs) and coffee-derived nanovesicles (CVs).

FIGS. 13A, 13B, 13C, and 13D show the viability of human BJ-5TA fibroblast (A, B) and mouse muscle C2C12 (C, D) cells tested by MTT assay after treatment with RVs and CVs 0.1, 0.5 and 1 mg/ml for 24 h.

FIGS. 14A, 14B, 14C, and 14D show the effects of the RDVs, RVs and CVs on H₂O₂ induced reactive oxygen species (ROS) and MDA production levels in fibroblast BJ-5TA cells (14A, 14B) and C2C12 (14C, 14D) cells7.

FIG. 15 shows collagen protein levels in human BJ-5TA fibroblast cells pretreated with RDVs, RVs and CVs 0.5 mg/ml in oxidative stress induced damage.

FIGS. 16A, 16B, 16C, and 16D show NRF2, COX2, FASN and GLUT4 protein levels in mouse muscle C2C12 cells pretreated with RVs and CVs 0.5 mg/ml in oxidative stress induced damage.

FIGS. 17A and 17B show cryogenic electron microscopy of the preferred composition of the present invention: 0.5-2 μm things with a sharp edge similar to a membrane, but different from a lipid bilayer. These PDVs are fully filled. Defined as microvesicles (17A) and 50-200 nm objects without sharp edge, defined as lipidic drops (17B).

FIG. 18 (comparative): Cryogenic electron microscopy of a composition obtained according to the described protocol 2, no vesicles are identified.

FIG. 19 (comparative): Cryogenic electron microscopy of a composition obtained according to the described protocol 2, very few vesicles are identified, while many protein bodies are identified.

FIG. 20A shows NTA graph of the sample obtained by applying the extraction protocol according to the invention on banana pulp and peel.

FIG. 20B shows NTA graph of the sample obtained by applying the extraction protocol according to the invention on rocket.

The object of the present invention is therefore a method for the production, purification and stabilization of plant-derived nanovesicles (PDVs), comprising the following steps:
a) Homogenization and sieving of a plant matrix to obtain a homogenate having a specific viscosity;
b) Separation of the juice from the solid residue present in the homogenate obtained in step a);
c) Isolation and purification of the PDVs contained in the extract thus obtained via ultrafiltration and/or Tangential Flow Filtration (TFF) and/or Size Exclusion Chromatography (SEC);
d) Stabilization of said nanovesicles and nanoparticles by spray-drying.

The homogenate obtained in said step a) is characterized by its viscosity, wherein this parameter has been evaluated by using a rotational viscometer (Viscometer Rotavisc me-vi, version: 1.02.004/1.11.004, IKA, Staufen, Germany). The viscosity of the samples was determined at a stable temperature of 25±1° C. The VOL-SP-6.7 spindle was able to read the viscosity of the complete set of emulsions. The viscosity readings were taken for 60 sec at 200.00 rpm rotation speed.

The method according to the present invention enables the production of plant-derived nanovesicles through mechanical processing of plant tissues.

The vesicles to which this invention refers, having unique properties as detailed below, are called PDVs (Plant-Derived nanovesicles). For the purposes of the present invention, “PDVs” denotes the product obtained according to the method claimed herein, said product comprising both nanovesicles and nanoparticles.

This method can be applied to selected plant matrix and waste raw materials.

In the context of the present invention, the term “plant matrix” designates any plant-derived material including, for example, leaves, stems, roots or flowers.

Preferably, said plant matrix is derived from a vegetable having a low sugar content.

According to the present invention, the term “low sugar content” refers to a sugar content lower than 20% by weight, preferably lower than 10% by weight, more preferably lower than 5% by weight with respect to the total weight of the plant.

Examples of such plant matrix include but are not limited to rocket, rosemary, coffee, artichoke, vine (Vitis vinifera), sage, passionflower or valerian. Preferably, the method according to the invention is applied to rocket, rosemary and coffee.

Step a) comprises subjecting the plant matrix to an extraction process using techniques well known to those skilled in the art, including, for example, Soxhlet, maceration, ultrasonic treatment, or mechanical agitation.

Preferably, it is macerated in a buffer solution at a temperature comprised between −20° C. and room temperature, more preferably at +4° C. for one night.

In an embodiment, said method comprises macerating the plant matrix overnight at +4° C. in Tris-HCl.

Optionally, the resulting homogenate can also be subjected to sonication in order to improve cell membrane disruption and cellular content release.

The intermediate homogenate is then subjected to sieving so as to reduce its viscosity through an initial removal of solid material corresponding to disrupted plant cell membranes.

The impact of the ultrasound technology and sieving on the viscosity of the resulting homogenate was studied and the results are presented in Example 1.

Therefore, according to a preferred embodiment, the resulting homogenate has a viscosity comprised between 1.5 mPa·s and 9.5 mPa·s, preferably between 2 mPa·s and 9 mPa·s.

In an embodiment, said homogenization step is carried out by blending at 200 Watt, 1′, at 4° C.

Subsequently, the solid material is separated from the extracted homogenate (step b)) to obtain an extract.

Different techniques can be used to separate the extract from the residual solid material, such as ultracentrifugation, TFF and/or differential centrifugation.

A preferred technique is differential centrifugation followed by TFF.

As it is well known to the skilled person, differential centrifugation involves the application of several cycles of increased centrifugal force for a given time in order to produce enriched fractions with decreased sedimentation speed.

According to the present invention, step b) consists of a number of cycles comprised between 2 and 5, preferably about 4 at an increasing centrifugal force between 500 g and 4500 g.

Preferably, each cycle lasts between 5 minutes and 45 minutes, and is performed at a temperature of approximately 4° C.

In an embodiment, said separation is obtained by subsequential centrifugation steps. In a preferred embodiment, said subsequential centrifugation steps are as follows:

$$700 g \times {10}^{′} at 4 ° C .$$

$$700 g \times {10}^{′} at 4 ° C .$$

$$1500 g \times {20}^{′} at 4 ° C .$$

At the end, the supernatant was collected and centrifuged at 3000 g×30′ at 4° C.

The enriched fractions thus collected are then subjected to ultrafiltration in step c) of the present method.

Any ultrafiltration technique can be used in the method of the present invention.

According to a preferred embodiment, the enriched fractions are filtered with the aid of one or more ultrafiltration membranes, preferably with the aid of two ultrafiltration membranes.

The ultrafiltration membranes useful according to the present invention have a molecular cutoff of 100 kDa and 10 kDa and are used sequentially.

In an embodiment, the following cut off have been subsequently used:
Cut off 100 at 4000 g×30′ at 4° C.
Cut off 10 4000 g×10′ at 4° C.

After passage over the aforementioned ultrafiltration membranes, the plant-derived nanovesicles thus obtained are isolated and subsequently purified in step c) with techniques known to those skilled in the art.

Examples of such techniques are ultracentrifugation, TFF, and SEC.

In an embodiment, said TFF is operated under controlled pressure, using a 10 kDa ceramic membrane filter.

Preferably said purification in step c) is carried out with SEC.

For such separation, an isocratic elution with an aqueous solvent, preferably a buffer, more preferably Tris-HCl, is preferably used. In an embodiment, SEC 35 nm eluted in Tris-HCl is performed.

In a preferred embodiment, the column is eluted with 20 mM Tris-HCl buffer, pH 7.2. The sample is loaded onto the column and, as the buffer (mobile phase) flows through, it drives the sample through the column, which is packed with a solid resin (stationary phase). The resin forms pores that enable gradual fractionation of the sample based on size. PDVs according to the invention are eluted in the first three fractions collected from the column. In this way it is possible to separate the fraction containing the PDVs, which elute as the head fraction of the chromatographic column with an eluent volume comprised between 2.5 and 12.5 mL, while the smaller sized compounds elute in a second moment.

The method of the present invention further comprises a spray-drying step d) of the fraction containing the PDVs.

According to a preferred embodiment, the spray drying is carried out with a Format 4 M8 type apparatus (ProCepT, Belgium) with the operating conditions reported in Example 1.

This step makes it possible to obtain PDVs in powder form and therefore particularly stable for long-term storage.

The Applicant of the present invention has surprisingly found that the above method allows to obtain a composition comprising nanovesicles having a specific particle size distribution.

The distribution of the composition obtained with the method of the present invention was measured with a Format 4 M8 apparatus (ProCepT, Belgium). This distribution provided a particle diameter comprised between 80 nm and 280 nm.

No change in size was observed before and after the spray-drying carried out in step d) of the present invention.

The method according to the invention involves the use of 20 mM Tris-HCl buffer at pH 7.2, rather than PBS, as used in state-of-the-art methods. The production of PDVs is made possible by an initial processing step of the plant matrix, which induces the generation of PDVs as naturally bio-formulated nanostructures.

The process then includes a series of sample cleaning phases (removal of coarse material) and a gradual isolation of the PDVs. The method according to the invention does not involve the use of ultracentrifugation, a technique that leads to a dramatic reduction in nanovesicles yield, loss of structural integrity, and, consequently, a significant decrease in bioactivity.

The final purification step uses a SEC column with a 35 nm cutoff, rather than 70 nm, enabling the isolation of smaller-sized PDVs.

Preferably, the nanovesicles of the composition of the present invention have the following particle distribution, as indicated in table 1 below:

TABLE 1

| D₁₀ | D₅₀ | D₉₀ |
|---|---|---|
| 80-120 nm | 120-180 nm | 210-280 nm |

D₁₀ refers to the diameter below which 10% of the particles are present; D₅₀ refers to the median diameter, i.e. the diameter below which 50% of the particles are present; D₉₀ refers to the diameter below which 90% of the particles are present.

Preferably said nanovesicles have a median diameter between 80 nm and 280 nm, more preferably between 120 nm and 180 nm.

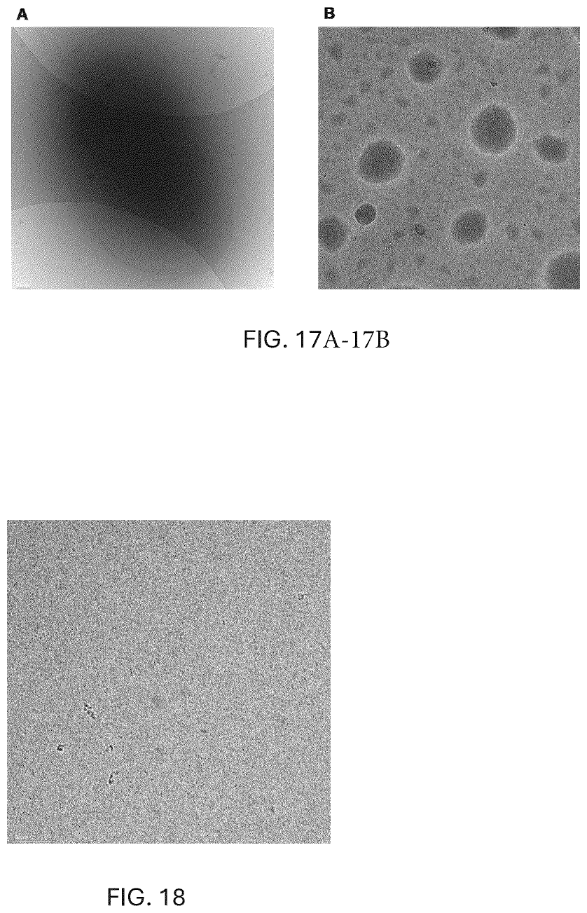

The Applicant of the present invention has also found that, further to the nanovesicles, said PDVs also comprises lipidic particles (see FIG. 17), having an average particle diameter in the nanometer range.

Preferably, said lipidic particles have an average particle diameter comprised between 50 and 200 nm.

Therefore, a further object of the present invention is represented by the composition PDVs, comprising plant-derived nanovesicles obtained by the present method and lipid nanoparticles.

Unlike the extracellular nanovesicles known in the literature which are pre-existing in the cellular structure, the nanovesicles of the present invention are mechanically induced and produced, purified and stabilized by the method described above.

They differ from the extracellular vesicles already known for several parameters:
The produced quantities: The extracellular apoplastic vesicles, naturally produced by the cells, are released in low quantities and the obtention of an enriched fraction containing them is particularly difficult;
The dimensions: The naturally produced extracellular vesicles have a diameter that does not depend on the technology used to purify them;
The composition: In the case of extracellular vesicles, the composition is actively controlled by the cell, while in the vesicles of the present invention, the composition reflects the phytochemical profile of the matrix which is bio-formulated when the vesicles are formed.

The PDVs according to the invention exert their biological activity through the release, into the target cell, of the phytocomplex contained therein, said phytocomplex comprising microRNA (miRNA) and secondary metabolites, such as polyphenols, the relative amounts of which vary depending on the plant matrix.

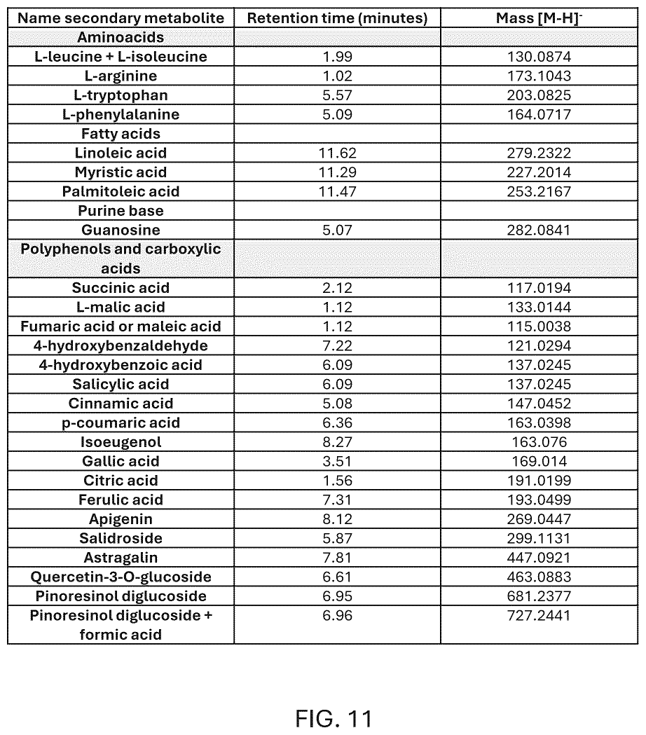

In the case of rocket, the phytocomplex includes 26 different secondary metabolites belonging to 4 different chemical classes of molecules: 15.4% of amino acids, 11.5% of fatty acids, 3.8% of purine base and 69.3% of polyphenols and carboxylic acids. The individual secondary metabolites present in the phytocomplex were isolated and identified using HR-HPLC-MS (High-Resolution High-Performance Liquid Chromatography Mass Spectrometry) and are shown in FIG. 11.

Furthermore, the phytocomplex isolated from rocket contains different families of microRNAs, which have been reported in the table of example 6.

The PDVs therefore function as a vehicle for biologically active molecules which, if administered without the aid of the vesicles, demonstrate lower biological activity.

As described in the experimental part of the present application, in particular in examples 3-6, the aforementioned PDVs are very effective in the treatment of various pathologies, demonstrating a significant hypocholesterolemic, hypolipidemic and hypoglycemic effect. They also show a strong antioxidant effect by reducing the intracellular malondialdehyde production (see example 7), thus also improving collagen production in fibroblasts (see example 8).

Furthermore, the present invention is directed to a pharmaceutical, nutraceutical or cosmetic formulation containing the composition prepared according to the present invention and at least one physiologically acceptable excipient.

A further object of the present invention is therefore represented by plant-derived nanovesicles and/or by a pharmaceutical, nutraceutical or cosmetic formulation that contains them for hypocholesterolemic, hypolipidemic, hypoglycemic, antiageing, and antioxidant use.

Although the invention has been described in its characteristic aspects, modifications and equivalents which are apparent to those skilled in the art are included in the following invention.

The present invention will now be illustrated by means of some examples, which should not be seen as limiting the scope of the invention.

### EXPERIMENTAL PART

### Example 1: Methods of Isolation and Purification of Vesicles of Plant Origin

### Sample Preparation.

To obtain rocket-derived nanovesicles (RDVs), ready to eat commercially available rocket leaves were carefully washed with deionized water and then macerated at 4° C. overnight in 1:1 ratio Tris-HCl 20 mM pH 7.2. The next day, 100 g of rocket leaves were blended with a blender with steel cross-shaped blades for 1 minute at 250 Watt keeping the sample as cool as possible to avoid the matrix degradation and encourage the vesicles formation through the membranes rearrangement.

The effect of ultrasound treatment on homogenate viscosity was evaluated by sonicating a portion of the homogenate in an ultrasonic bath, 37 kHz for 5 minutes. Then, both the samples were sieved, and the viscosity study was assessed in all the samples. The study was performed by using a rotational viscometer (Viscometer Rotavisc me-vi, version: 1.02.004/1.11.004, IKA, Staufen, Germany). The viscosity of the formulations was determined at a stable temperature of 25±1° C. The VOL-SP-6.7 spindle was able to read the viscosity of the complete set of emulsions. The viscosity readings were taken for 60 sec at 200.00 rpm rotation speed. The results were expressed as the average of three different samples and are reported in table 2 below.

TABLE 2

| | Viscosity (mPa · s) ± standard |
|---|---|
| Sample | deviation (n = 3) |
| A | 13.3 ± 2.7 |
| B | 21.6 ± 5.9 |
| A1 | 5.8 ± 0.2 |
| B1 | 4.2 ± 0.0 |

A = Rocket leaves subjected to maceration and blending only
B = Rocket leaves subjected to maceration and subsequent sonication
A1 = Rocket leaves subjected to maceration and subsequently sieved
B1 = Rocket leaves subjected to maceration, sonication and subsequently sieved

The same procedure was applied on rosemary leaves (50 g) and coffee powder (50 g).

The results are reported in table 3 below.

TABLE 3

| | | mPa*s [Mean ± standard |
|---|---|---|
| | Sample | deviation (n = 3)] |
| Rosemary | A | 411.7 ± 12.6 |
| | B | 237.5 ± 13.1 |
| | A1 | 8.4 ± 2.7 |
| | B1 | 2.6 ± 0.2 |
| Coffee | A | 135.8 ± 17.6 |
| | B | 71.7 ± 27.5 |
| | A1 | 3.9 ± 0.2 |
| | B1 | 7.6 ± 0.8 |

### Production of Rocket-Derived Nanovesicles

For comparison purposes, rocket-derived nanovesicles were obtained by means of three different protocols: the first consisting of ultracentrifugation (UC) as the reference technique, the second by differential centrifugation (DC) followed by ultrafiltration (UF) and size exclusion chromatography (SEC), and the third by DC followed by the tangential flow filtration.

### 1. Production by Ultracentrifugation

After the mixing step, the homogenate was resuspended in 20 mM Tris-HCl pH 7.2 and then filtered through a 100 μm filter to remove coarse debris. The sample was centrifuged as follows:
500 rpm (750 g) for 10 min. The supernatant is recovered for subsequent centrifugation and the pellet is discarded.
1500 rpm (2000 g) for 20 min. The supernatant is recovered for subsequent centrifuging and the pellet is discarded.
3000 rpm (4500 g) for 30 min.
10000 rpm for 1 h in Beckman Coulter (Type 50.2 Ti rotor) 4° C. The supernatant is recovered for subsequent centrifugation and the pellet is discarded.
28800 rpm for 3 h in Beckman Coulter (Type 50.2 Ti Rotor). The pellet is resuspended in 500 μl of sterile 1×PBS.

### 2. Production by Differential Centrifugation, Ultrafiltration, and Size Exclusion Chromatography

In the second method, after sample processing, the homogenate was sieved to remove coarse plant material. The sonicated and non-sonicated sieved homogenates (samples A1 and B1) were subjected to differential centrifugation to remove large debris and plant material as follows:
Centrifuge at 700 g for 10 minutes at 4° C. The pellet was discarded and the supernatant collected for the subsequent centrifugation step
Centrifuge at 700 g for 10 minutes at 4° C. The pellet was discarded and the supernatant collected for the subsequent centrifugation step
Centrifuge at 1500 g for 20 minutes at 4° C. The pellet was discarded and the supernatant collected for the subsequent centrifugation step
Centrifuge at 3000 g for 30 minutes at 4° C. The pellet was discarded, and the supernatant was collected.

The supernatant was ultrafiltered through a filter device (Amicon® Ultra 100K device—100,000 MWCO) by applying a centrifuge at 4000 g for 30 minutes at 4° C. The filtrate was ultrafiltered with an Amicon® Ultra 10K—10,000 MWCO device by applying a centrifuge at 4000 g for 10 minutes at 4° C.

### 2.1 Purification of Rocket-Derived Nanovesicles by Size Exclusion Chromatography.

The obtained sample was eluted in size exclusion chromatography (SEC) columns to purify the sample and isolate the food-derived nanovesicles by removing all possible interferents. SEC columns separate particles based on their size as they pass through the column packed with a porous stationary phase. When molecules enter the stationary phase, smaller particles become trapped in the pores and their exit from the column is delayed. Therefore, sequential fractions are collected. The particles will be distributed among the fractions based on their size, with the largest particles exiting the column first and the smallest particles exiting the column last. In particular, the sample (500 μl) was loaded at the head of the column (qEVoriginal/35 nm Legacy Column) and eluted with a buffer (20 mM TrisHCl pH 7.2). At the end of the column, the first three vesicle-enriched fractions (500 μl each) were carefully collected.

In addition, NTA analyses were conducted on samples A, B, A1 and B1 to study their PDVs concentration and particle size distribution.

Particle size distribution and concentration were analyzed by a nanoparticle tracking analysis (NTA) using a NanoSight NS300 (Malvern Panalytical, Malvern, UK) equipped with a blue laser (404 nm, 70 mV) and sCMOS camera. Before the analysis, the samples were diluted in HPLC-grade water to reach the ideal particle concentration range in terms of particles/frame (20-120 particles/frame). Temperature was held constant at 25° C. during the experiment, and for each sample, five 60 s videos were recorded and, subsequently, analyzed using NTA software 3.0 (NanoSight, Malvern Panalytical, Malvern, UK).

| Sample | Concentration (PDVs/mL) |
|---|---|
| A | 5.33 ± 0.68 × 10⁸ |
| B | 8.79 ± 1.47 × 10⁷ |
| A1 | 2.85 ± 0.31 × 10⁷ |
| B1 | 3.07 ± 0.29 × 10⁷ |

The results show sonication reduces the nanoparticles concentration/ml (8.79±1.47×10⁷ nanoparticles/ml) compared to the non-sonicated sample (5.33±0.68×10⁸ nanoparticles/ml).

Qualitatively, the size distribution of the vesicles after sonication appears to be more heterogeneous than in the respective non-sonicated samples. In the sample A, only one peak is observed around 88 nm.

2.2 Stabilization of Nanovesicles Extracted from Rocket by Spray Drying

After the collection of the vesicle-enriched fractions, the spray drying technique was applied to stabilize the sample obtaining powder. This technology is used to transform liquids (solutions, emulsions, suspensions) into solid powders. Its main applications concern the food and chemical industries and aim to increase the preservation and stability of ingredients. Additionally, spray drying can be used for specific applications in the formulation of pharmaceutical products.

The following samples were tested:

| Formulation | Physical state | Purification method |
|---|---|---|
| 1 | Solution | Ultracentrifugation |
| 2 | Solution | Ultracentrifugation |
| 3 | Solution | Ultracentrifugation |
| 4 | Solution | SEC (column 1) |
| 5 | Solution | SEC (column 2) |
| 6 | Dried powder after spray | Ultracentrifugation |
| | drying | |
| 7 | Dried powder after spray | SEC (column 1) |
| | drying | |

Formulation 6 was prepared using the spray drying process described below from Formulation 1.

Formulation 7 was prepared by the spray drying process described below from formulation 4.

Column 2 is a column used mainly for the purification of nucleic acids (Amersham NAP-5, Cytiva) while column 1 is specific for the purification of animal-derived vesicles (qEVoriginal/35 nm Legacy Column) here optimized for plant-derived nanovesicles.

### Methods

### Spray Drying

Aliquots of formulations 1 and 4 were diluted in a trehalose solution to obtain a final sugar concentration of 10% w/v. The obtained solution was then sprayed through a two-fluid nozzle, operating in co-current, of a Format 4 M8 apparatus (ProCepT, Belgium). The process parameters were set as reported in table 4. The dried powders were separated from the drying air in the cyclone (outlet temperature=37-40° C.) and deposited in the collector.

TABLE 4
spray drying parameters

| | | |
|---|---|---|
| Power flow | 5 | mL/min |
| Nozzle diameter | 0.4 | mm |
| Nozzle pressure | 1.7 | atm |
| Air speed | 0.3 | m³/min |
| Air inlet temperature | 120° | C. |
| Δ Pressure | 70 | mbar |

### Dynamic Light Dispersion (DLS)

The Z-average diameter (Dh) and polydispersity index (PDI) of the samples were evaluated by photon correlation spectroscopy using dynamic light scattering (DLS, Zetasizer Nano ZS, Malvern Instrument, Malvern, UK), equipped with a backscattered light detector, operating in dual angle mode (173°, 12.8°) and at 25° C. Prior to analysis, samples were diluted in HPLC grade water to reach the ideal particle concentration range. Results calculated using Dispersion Technology software (Malvern Instruments, Malvern, UK) are reported as an intensity distribution.

### Analysis for Nanoparticle Monitoring

Particle size distribution and concentration were analyzed by nanoparticle tracking analysis (NTA) using a NanoSight NS300 (Malvern Panalytical, Malvern, UK) equipped with a blue laser (404 nm, 70 mV) and an sCMOS camera. Prior to analysis, samples were diluted in HPLC grade water to reach the ideal particle concentration range in terms of particles/frame (20-120 particles/frame). The temperature was kept constant at 25° C. throughout the experiment, and five 60-s videos were recorded for each sample and subsequently analyzed using NTA 3.0 software (NanoSight, Malvern Panalytical, Malvern, UK).

### Results

Spray-dried plant-derived nanovesicle powders were obtained with a yield greater than 65%, regardless of the type of vesicle source and/or purification method (Formulation 1 or Formulation 4).

The size distribution patterns of the plant-derived nanovesicle formulations obtained by DLS and NTA are shown in tables 5 and 6. As can be observed in table 5, the plant-derived nanovesicle solutions were found to be highly polydisperse. However, it is interesting to note that the purification method affects the particle size pattern and concentration. Comparing formulations 1-3 and formulations 4-5 (table 5), it can be seen that:
a) ultracentrifugation seems less reproducible in terms of final hydrodynamic diameter (Dh) and PDI compared to UF+SEC;
b) the predominant particle population is 130-437 nm in the case of UF+SEC, rises up to 4000 nm in the case of ultracentrifugation;
c) all ultracentrifuged samples contained particles/aggregates capable of being detected by DLS in forward diffusion mode.

In fact, DLS analyzes in dual angle mode were performed to highlight the presence of aggregates. Performing measurements in both the backward diffusion (173°) and forward diffusion (12.8°) directions allowed us to monitor the onset of protein/particle aggregation. DLS uses intensity fluctuations of scattered light to measure the size and size distribution of proteins in solution. As the size of the protein/particle or their aggregate increases to become greater than ≈λ/10 and approaches the wavelength (λ) of the laser, the scattered intensity begins to increase at 12.8°. In light of this, it is possible to conclude that no aggregates are present in the samples purified by SEC (Formulations 4, 5, 7); while the aggregates are detected at 12.8° in all ultracentrifuged samples (Formulations 1, 2, 3, 6).

On the other hand, it is worth noting that a decrease in particle concentration is observable in the sample purified by UF+SEC compared to samples purified by ultracentrifugation (Formulation 3 compared to Formulation 4; table 6).

Focusing on SEC samples, the type of SEC column selected appears to have an impact on the size distribution pattern of plant-derived nanovesicles. In particular, the use of column 1 enables the obtainment of plant-derived nanovesicles smaller in size than those from column 2 (Formulation 4 compared to Formulation 5; table 5). No significant difference was observed in terms of polydispersity index.

Regarding spray drying, the overall results highlight that the preparation process has an impact on the size distribution model and not on the concentration of plant-derived nanovesicles, regardless of the purification method. Indeed, the average hydrodynamic diameter of the vesicles remains comparable in both samples purified by ultracentrifugation (Formulation 1 compared to Formulation 6; table 5) and those obtained by UF+SEC (Formulation 4 compared to Formulation 7; tables 5 and 6).

TABLE 5
Results DLS

| Form. | Sample preparation | Angle (°) | Dₕ (nm) | PDI |
|---|---|---|---|---|
| Form. 1 | Dilution 1:20 | 173° | 314 ± 117 | 0.724 ± 0.024 |
| | | 12.8° | 636 ± 23 | 0.873 |
| Form. 2 | Dilution 1:20 | 173° | 4400 ± 518 | 0.242 ± 0.007 |
| | | 12.8° | 600 ± 32 | 0.509 ± 0.060 |
| | Dilution 1:200 | 173° | 1707 ± 441 | 0.447 ± 0.299 |
| | | 12.8° | 1211 ± 265 | 1.000 ± 0.000 |
| Form. 3 | Dilution 1:20 | 173° | 179 ± 16 | 0.756 ± 0.060 |
| | | 12.8° | 426 ± 7 | 0.142 ± 0.024 |
| Form. 4 | Dilution 1:20 | 173° | 138 ± 12 | 0.283 ± 0.224 |
| | | 12.8° | — | — |
| Form. 5 | Dilution 1:20 | 173° | 437 ± 74 | 0.434 ± 0.131 |
| | | 12.8° | — | — |
| Form. 6 | Solution | 173° | 440 ± 126 | 0.732 ± 0.100 |
| | 18 mg/ml of | 12.8° | 632 ± 49 | 0.788 ± 0.273 |
| | dried powder(^{a}) (*) | | | |
| | diluted 1:10 | | | |
| Form. 7 | Solution | 173° | 131 ± 11 | 0.431 ± 0.147 |
| | 18 mg/ml of | 12.8° | — | — |
| | dried powder(^{a}) (*) | | | |
| | diluted 1:10 | | | |

(^{a})The vesicle concentration in a solution of 18 mg/ml spray-dried powder was estimated to be similar to the vesicle concentration in Formulation 1.

TABLE 6
Results NTA

| | Sample | D₁₀ | D₅₀ | D₉₀ | Particles/ | |
|---|---|---|---|---|---|---|
| Form. | preparation | (nm) | (nm) | (nm) | mL^{(b)} | |
| Form. 3 | Dilution 1:900 | 104 ± 3 | 142 ± 2 | 218 ± 3 | 5.8 | (±0.2) * 10¹⁰ |
| Form. 4 | Dilution 1:3 | 117 ± 20 | 170 ± 10 | 239 ± 20 | 1.9 | (±0.8) * 10^{7(c)} |
| Form. 7 | Dilution 1:3 | 87 ± 2 | 122 ± 2 | 214 ± 2 | 6.7 | (±0.2) * 10⁸ |

^{(b)}Concentration referred to the undiluted solution
^{(c)}The results may not be reliable as the particles/frames were less than 20.

In light of the results, formulation 7 containing the rocket-derived vesicles obtained with the second protocol (i.e. differential centrifugation, ultrafiltration, SEC chromatography and spray drying) was used for the evaluation of the biological activities on Caco-2 and HepG2 cells.

### 3. Production by DC+Tangential Flow Filtration

Tangential flow filtration (TFF) is a type of filtration method used in various industries, including biopharmaceuticals, food and beverage, and wastewater treatment. Unlike traditional filtration methods where the fluid being filtered flows directly perpendicular to the filter membrane (called dead-end filtration), TFF involves the flow of the fluid parallel to the filter membrane.

In TFF, the fluid to be filtered is pumped tangentially across the surface of a porous membrane. As the fluid flows along the membrane, two streams are generated: the retentate, which contains the larger particles or molecules that are unable to pass through the membrane, and the permeate (also known as filtrate), which contains the smaller particles or molecules that have passed through the membrane.

In our research, we have processed rocket homogenate (after homogenization and sieving steps followed by differential centrifugation steps) and used a 10 kDa membrane to recover the permeate. Then 2, 3, and 4 dialysis steps were performed by adding each time deionized water/20 mM TrisHCl pH 7.2, respectively. The first permeate and the other dialysis permeates were pooled in order to obtain a single solution that was stabilized by spray drying technique. The following table 7 reports the NTA results obtained on rocket-derived vesicles.

TABLE 7
Results NTA

| | | | | | | | Concentration | |
|---|---|---|---|---|---|---|---|---|
| | D10 (nm) | | D50 (nm) | | D90 (nm) | | (particles/mL) | |
| | | Standard | | Standard | | Standard | | Standard |
| Sample | Mean | error | Mean | error | Mean | error | Mean | error |
| powder | 129 | 1.1 | 189.9 | 3.1 | 281.3 | 3.5 | 3.0.10⁸ | 2.3.10⁷ |

### Example 2: Isolation and Purification of Plant-Derived Vesicles (PDVs) from New Food Matrices: Coffee-Derived Vesicles (CVs) and Rosemary-Derived Vesicles (RVs)

### Sample Processing

To obtain CVs and RVs, commercially available coffee powder (50 g) and rosemary leaves (50 g) were macerated, separately, at 4° C. overnight in 1:1 Tris-HCl 20 mM pH 7.2. The next day, the leaves and the powder were blended for few minutes to mimic the chewing step that occurs in the mouth during the first step of the digestive process. The next day, the samples were blended with a blender with steel cross-shaped blades for 1 minute at 250 Watt keeping the sample as cool as possible to avoid the matrix degradation and encourage the vesicles formation through the membrane's rearrangement. The samples were sieved to recover the liquid filtrate.

### CVs and RVs Isolation and Purification by Differential Centrifugation and Ultrafiltration

After the sieving step, CVs and RVs were isolated and purified by differential centrifugation (dUC) followed by ultrafiltration (UF) step, respectively.

The homogenates obtained were subjected to differential centrifugation to remove large debris and plant material as follows:
Centrifuge at 700 g×10 minutes at 4° C. The pellet was discarded, and the supernatant collected for the next centrifugation step;
Centrifuge at 700 g×10 minutes at 4° C. The pellet was discarded, and the supernatant collected for the next centrifugation step;
Centrifuge at 1500 g×20 minutes at 4° C. The pellet was discarded, and the supernatant collected for the next centrifugation step;
Centrifuge at 3000 g for 30 minutes at 4° C. The pellet was discarded, and the supernatant was collected.

The supernatant was ultra-filtrated by a filter device (Amicon® Ultra 100K device—10,000 MWCO) applying a centrifuge at 4000 g×30′ at 4° C. The retentates were discarded and the filtrate kept for the following analysis.

NTA and DLS analyses were conducted on 10 kDa cut off samples to study the samples PDVs concentration and size.

### Nanoparticle Tracking Analysis (NTA)

Particle size distribution and concentration were analyzed by a nanoparticle tracking analysis (NTA) using a NanoSight NS300 (Malvern Panalytical, Malvern, UK) equipped with a blue laser (404 nm, 70 mV) and sCMOS camera. Before the analysis, the samples were diluted in HPLC-grade water to reach the ideal particle concentration range in terms of particles/frame (20-120 particles/frame). Temperature was held constant at 25° C. during the experiment, and for each sample, five 60 s videos were recorded and, subsequently, analyzed using NTA software 3.0 (NanoSight, Malvern Panalytical, Malvern, UK).

### Dynamic Light Scattering

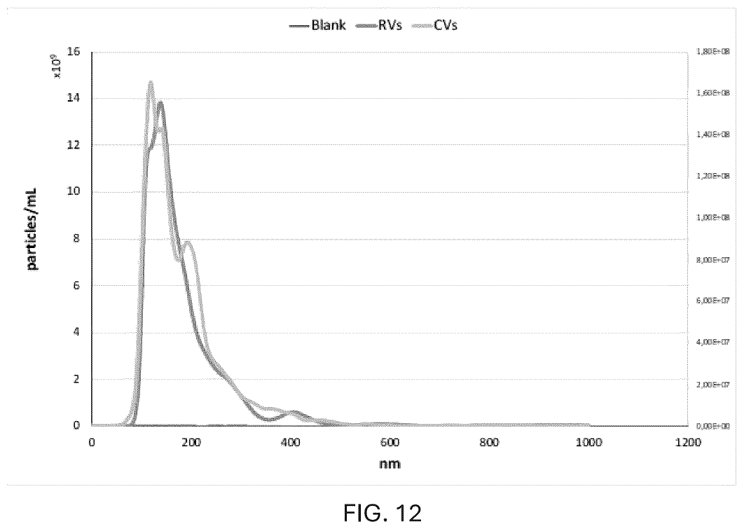

The Z-average diameter (Dh) and the polydispersity index (PDI) of samples were evaluated by photon correlation spectroscopy using a dynamic light scatter (DLS, Zetasizer Nano ZS, Malvern Instrument, Malvern, UK), equipped with a backscattered light detector, operating at dual angle (173°, 12.8°) mode and at 25° C. Before the analysis, the samples were diluted in HPLC-grade water to reach the ideal particle concentration range. The results calculated using the Dispersion Technology Software (Malvern Instruments, Malvern, UK) are reported as intensity distribution (see FIG. 12).

| | | DLS | | | | | | NTA | | | | |
|---|---|---|---|---|---|---|---|---|---|---|---|---|
| Sample | Angle | Dh (nm) | | PDI | | DCR (kcps) | | D10 | D50 | D90 | Conc/mL | |
| CV | 173° | 300 | 15 | 0.351 | 0.061 | 5473 | 207 | 109.3 | 159.2 | 277 | 1.74 × 10¹⁰ | 4.34 × 10⁸ |
| | 12.8° | 593 | 69 | 0.923 | 0.134 | 6052 | 1077 | | | | | |
| RV | 173° | 222 | 25 | 0.479 | 0.059 | 16702 | 150 | 112.6 | 156.1 | 268.9 | 1.37 × 10¹² | 3.23 × 10¹⁰ |
| | 12.8° | 565 | 74 | 0.251 | 0.226 | 10124 | 1388 | | | | | |

### Example 3: Evaluation of Intestinal Trans-Epithelial Transport of Vesicles Extracted from Rocket onto Intestinal Caco-2 Cells

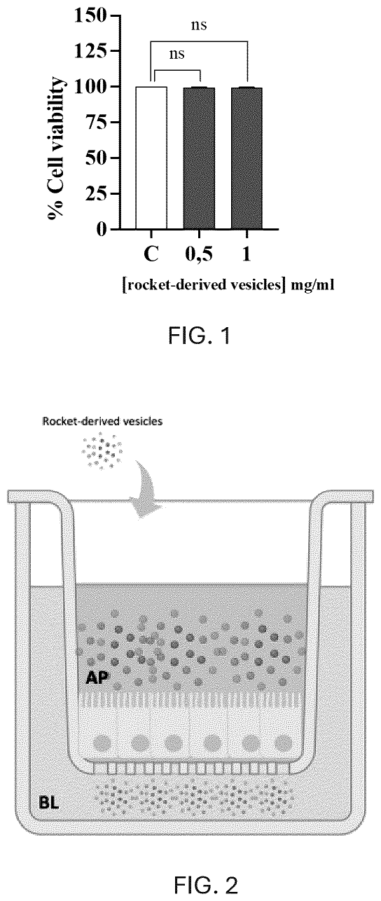

Intestinal vesicle transport was assessed using differentiated Caco-2 cells as a model of mature enterocytes. The Caco-2 cell line has been widely used as a model of the intestinal epithelial barrier and under appropriate conditions on a Transwell membrane system, the cells develop tight junctions, polarize, and acquire intestinal microvilli at the apical level where transport and metabolism of proteins occur. xenobiotics, since they express both phase I and phase II enzymes. Before the evaluation of intestinal transepithelial transport, MTT experiments were performed on HepG2 cells treated with vesicles extracted from rocket. The results show that the vesicles extracted from rocket have no cytotoxic effect for concentrations of 0.5 and 1 mg/mL (FIG. 1).

Before performing the intestinal transepithelial transport experiment, the vesicles were fluorescently labeled with Alexa Fluor 488 dye. Then, the transport of rocket-derived vesicles by mature Caco-2 cells was analyzed by loading the apical compartment with vesicles extracted from rocket (1 mg/mL) in the apical transport solution (500 μL) and the basolateral compartment with the basolateral transport solution (700 μL) (FIG. 2).

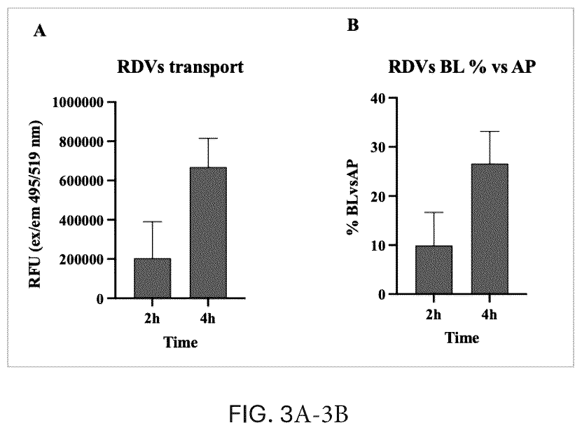

The plates were incubated at 37° C. and the basolateral solutions were collected at different times (2 and 4 hours). By measuring the fluorescence signal at 495 nm after excitation at 519 nm we determined the linear transport of the vesicle extracted from rocket (FIG. 3A). Furthermore, by comparing the percentage of fluorescence of the basolateral and apical solution, after 4 hours, it was observed that 26.6% of the vesicles in the apical chamber go to the basolateral chamber (FIG. 3B).

### Example 4: Hypocholesterolemic Activity of Rocket-Derived Vesicles

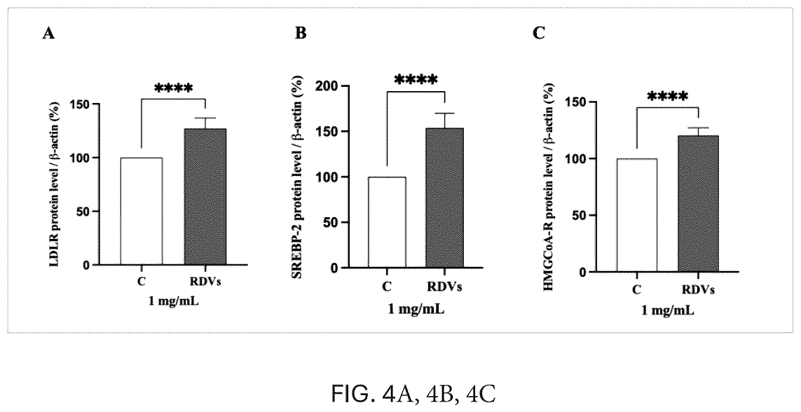

The ability of rocket-derived vesicles to modulate the LDL pathway was evaluated on HepG2 cells. LDLR expression is finely tuned by changes in intracellular cholesterol, and a transcription factor, known as sterol-responsive element-binding protein-2 (SREBP-2), plays a critical role in LDLR mRNA expression. The SREBP-2 isoform is responsible for the transcription of LDLR and HMGCOAR, and SREBP-2 maturation is regulated by intracellular cholesterol homeostasis (doi: 10.1038/343425a0). Therefore, HepG2 cells were treated with rocket-derived vesicles (1 mg/mL) for 24 hours. Rocket-derived vesicles up-regulated the protein level of the transcription factor SREBP-2 by 153.8%±16.14% (FIG. 4B) and increasing the SREBP-2 protein level led to an improvement in total levels of LDLR and HMGCoAR proteins up to 127.2±9.77% (FIG. 4A), 120.4±6.8% (FIG. 4C). Increased LDLR leads to increased clearance of plasma LDL cholesterol.

Furthermore, treatment with rocket-derived vesicles leads to the activation of the AMPK pathway and the consequent significant increase in the phosphorylation levels of HMGCOAR (serine 872, AMPK phosphorylation site) up to 166.5±23.3% (FIG. 5B). This result is consistent with the enhancement of AMPK (threonine 172) phosphorylation up to 144.6±11.25% (FIG. 5A).

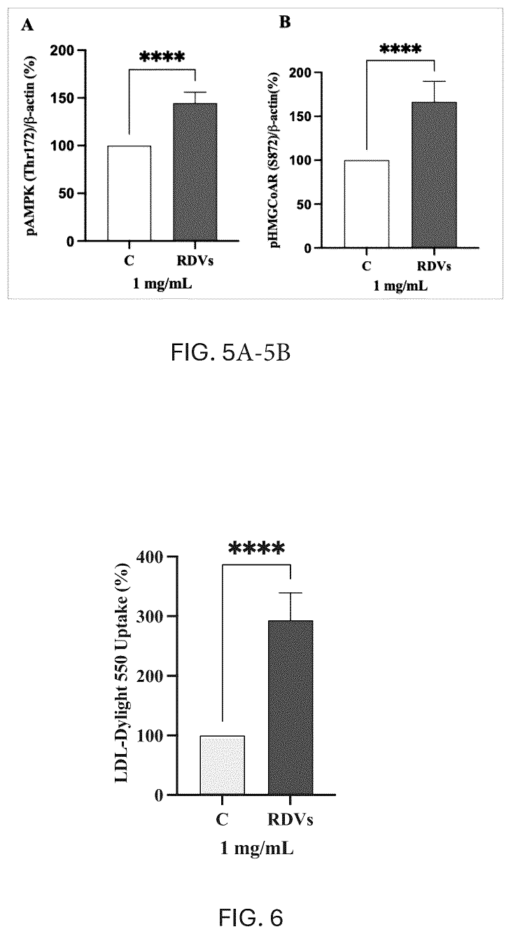

Furthermore, functional investigations were conducted with the aim of evaluating the ability of rocket-derived vesicles to modulate the ability of HepG2 to absorb extracellular LDL. Indeed, vesicles extracted from rocket (1 mg/mL) improved the ability of HepG2 cells to absorb LDL-Dylight 550 up to 293.5±45.65% (FIG. 6).

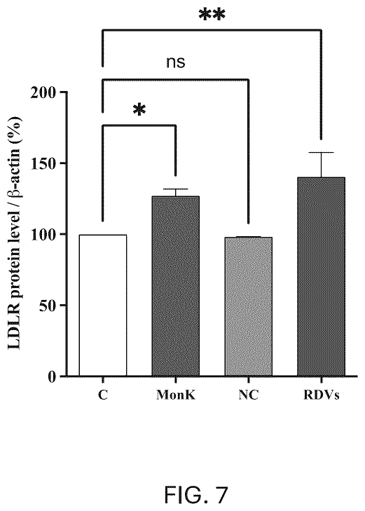

To evaluate the ability of the rocket-derived vesicle to inhibit HMGCoAR activity, a preliminary in vitro assay was performed. Since rocket-derived vesicles do not show effects on the direct inhibition of HMGCoAR in vitro, we hypothesized that the structure of the aforementioned vesicles is an important factor for their uptake by cells. After uptake, the rocket-derived vesicles exert a regulatory activity at an intracellular level. To investigate this hypothesis, those vesicles were lysed to disrupt the bilayer structure using RIPA+NP40% buffer. Then, HepG2 cells were treated with Monacolin K, rocket-derived vesicles, and lysed rocket-derived vesicles. Immunoblotting experiments show that unlike rocket-derived vesicles, no increase in the total LDLR protein level is observed for lysed rocket-derived vesicles (FIG. 7).

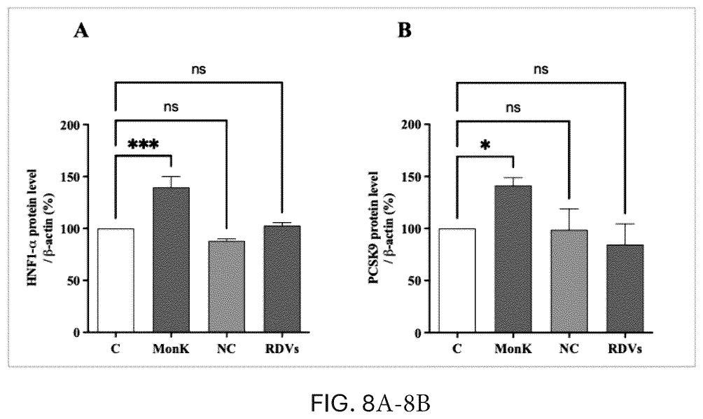

Indeed, rocket-derived vesicles were unable to modulate mature PCSK9 protein levels and were also ineffective on the activation of HNF1-α, the PCSK9 transcription factor (FIGS. 8A, 8B). In contrast, Monacolin K (1 μM), upon direct activation of HNF1-α up to 139.6±10.5% (p<0.001), significantly increased the PCSK9 protein level up to 141.3±7.6% (p)<0.05) (FIGS. 8A, 8B).

To note, the structural integrity of the vesicles is essential to their activity. This was proved by damaging rocket-derived vesicles by sonication for a few minutes. In FIG. 7, the lysed sample (NC) does not exert any activity.

### Example 5: Hypolipidemic Activity of Rocket-Derived Vesicles

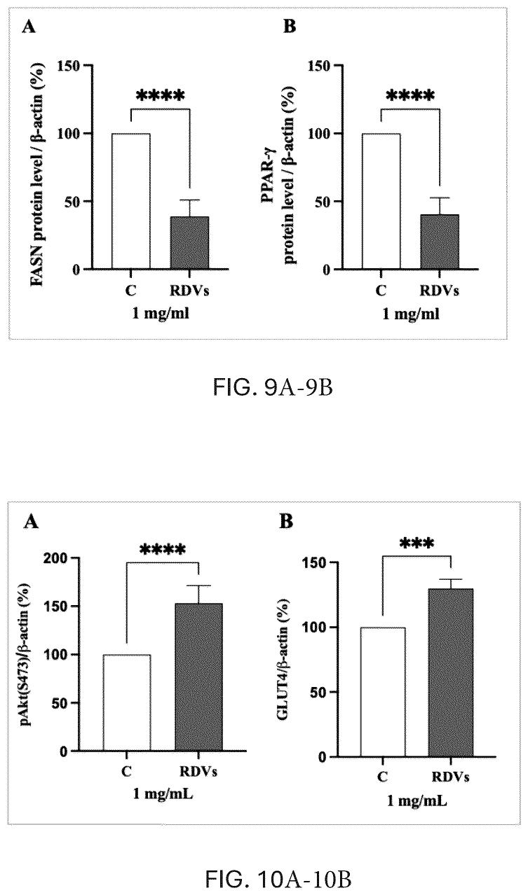

In light of the results of the hypocholesterolemic activity of rocket-derived vesicles, we investigated the hypolipidemic effect of the vesicles on HepG2 cells. In order to examine whether rocket-derived vesicles affect the pathways regulating lipid synthesis and accumulation in the liver, HepG2 was treated with those vesicles (1 mg/ml) and immunoblotting experiments were performed to detect the production of FASN and PPARγ proteins. Fatty acid synthesis is the key step for lipogenesis, which is responsible for the complete synthesis of palmitate from acetyl COA in the cytosol. Fatty acid synthase (FASN) is a key enzyme for lipogenesis and energy metabolism in vivo (doi: 10.1096/fasebj.8.15.8001737). Peroxisome proliferator-activated receptor γ (PPARγ) is an important transcription factor that regulates several transcriptional pathways related to adipogenesis and lipid metabolism. Accumulation of PPARγ in the liver induces hepatic steatosis through the activation of lipogenic genes, further enhancing hepatic lipogenesis and triglyceride synthesis (doi: 10.1074/jbc.M210062200). Therefore, according to the activation of AMPK pathway (FIG. 5A), immunoblotting experiments show the decrease of FASN protein levels up to 38.9±12.15% in HepG2 cells (FIG. 9A). Attenuated protein production of FASN indicates repression of de novo lipogenesis. Furthermore, the results show that PPARγ protein levels decrease by up to 40.5±12.2%. (FIG. 9B).

### Example 6: Antidiabetic Activity

Glucose uptake into cells is facilitated and tightly controlled by glucose transporters that show different expressions among different tissues (doi.org/10.1002/mnfr.201400850). GLUT4 is activated in response to insulin through activation of the phosphatidylinositol-4,5-bisphosphate 3-kinase (PI3K)-protein kinase B (Akt) pathway. In response to insulin, activation of Akt, through phosphorylation at serine 473, leads to a translocation of GLUT4 onto cell membranes. In order to examine whether rocket-derived vesicles affect Akt activation, HepG2 was treated with such vesicles (1 mg/ml) and immunoblotting experiments were performed detecting phosphorylated Akt upon production of serine 473 and GLUT4.

The results (FIG. 10) suggest that rocket-derived vesicles activate the Akt pathway with a significant increase in the levels of phosphorylated Akt and GLUT4 proteins up to 153±18.41% and 129.8±7.3% respectively.

### Example 7: Antioxidant Activity of Coffee and Rosemary Nanovesicles Tested on Fibroblasts and Myoblasts Cells

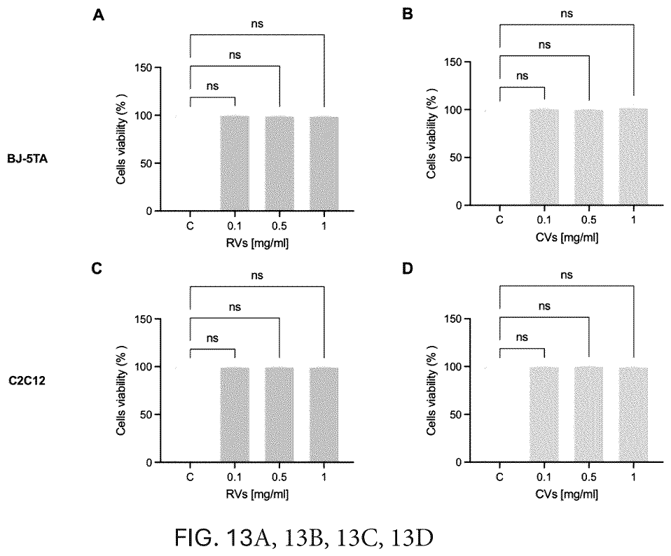

Before the assessment of antioxidant assays, to exclude cytotoxic effects, MTT assays were performed on BJ-5TA (human fibroblasts) and C2C12 (mouse myoblasts) cells. The cells were seeded on 96-well plate and treated for 24 hours with CVs and RVs at 0.1, 0.5 and 1 mg/ml, corresponding to 7.8×10⁶ nanovesicles/ml, 39×10⁶ nanovesicles/ml and 78×10⁶ nanovesicles/ml, respectively. The results (FIG. 13) clearly show the absence of negative effects on cells viability in both cell lines.

Considering the phytochemical profile of coffee and rosemary matrixes, we investigated the impact of both nanovesicles samples and the rocket-derived nanovesicles (RDVs) on ROS and MDA (malondialdehyde) production in BJ-5TA and C2C12 cells.

The results clearly show that all the samples are antioxidant in both the cell lines. In particular, based on these results, to evaluate whether the samples modulate the H₂O₂-induced ROS production, BJ-5TA and C2C12 cells were pre-treated 0.1 and 0.5 mg/mL RDVs, RVs and CVs overnight at 37° C. The next day, the same cells were treated with 200 μM in C2C12 and 20 μM H₂O₂ overnight at 37° C.

FIG. 13 clearly suggests that the treatment of cells with H₂O₂ alone produces a significant augmentation of intracellular ROS levels by 406±3.06% in fibroblasts and 1408±27.2% in myoblasts cells versus the control cells, which was attenuated by the pre-treatment with RDVs, RVs and CVs that reduced the H₂O₂ induced intracellular ROS.

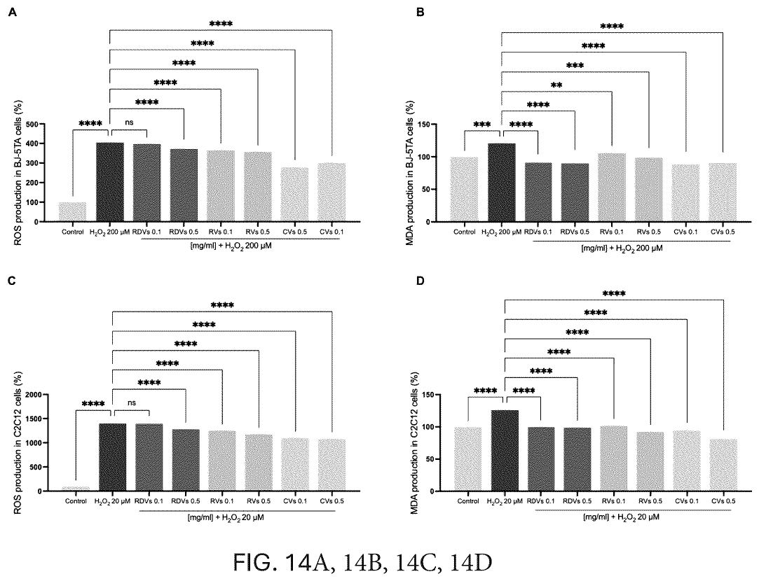

In particular, RDVs 0.5 mg/ml reduce ROS production up to 374.5±14.89% in fibroblast and 12915±5.92% in myoblasts, respectively; RVs 0.1 and 0.5 mg/ml reduce ROS production up to 366±14.534% and 358.7=0.863% in fibroblast and 1256±2.026% and 1181±11.76 in myoblasts; CVs 0.1 and 0.5 mg/ml reduce ROS production up to 302.4±4.878% and 279=6.853% in fibroblast and 1105±9.749% and 1084±13.91% in myoblasts, respectively, confirming that those vesicles can act as a natural antioxidant (FIG. 14, A and C).

Cellular membrane lipids are susceptible to oxidative damage, primarily by reactive oxygen species (ROS), leading to a specific chain reaction that produces end products such as malondialdehyde (MDA) and its related compounds, collectively referred to as TBA reactive substances (TBARS).

Considering these observations, the effectiveness of RDVs, RVs, and CVs in modulating H₂O₂-induced lipid peroxidation in fibroblasts and myoblasts was evaluated.

This evaluation involved measuring the reaction between the precursor of MDA and the TBA reagent to generate a fluorometric product (with excitation wavelength λex=532 nm and emission wavelength λₑₘ=553 nm), which is indicative of the quantity of TBARS (MDA present). In agreement with the observed increase of ROS after the H₂O₂ treatment, a significant increase of the lipid peroxidation was observed up to 121.2±0.9574% and 126.7±1.976% at cellular level in fibroblasts and myoblasts, respectively.

In addition, the pre-treatment of both the cell lines with all the samples determined a significant reduction of lipid peroxidation. FIGS. 14B and C clearly show that the MDA production was attenuated by the pre-treatment with RDVs, RVs and CVs.

In particular, RDVs 0.1 and 0.5 mg/ml reduce MDA production up to 91.43±3.11% and 90.48±10.94% in fibroblasts and 100.3±0.9204% and 99.50±1.329 in myoblasts, respectively; RVs 0.1 and 0.5 mg/ml reduce MDA production up to 105.8±1.880% and 99.05.±0.5.387% in fibroblasts and 101.9.±1.47% and 92.52±1.51 in myoblasts; CVs 0.1 and 0.5 mg/ml reduce MDA production up to 88.92±9.622% and 91.04±6.557% in fibroblasts and 94.42±2.92% and 81.58±4.24% in myoblasts, respectively (FIGS. 13A and C).

Since the lipid peroxidation is a validated marker of oxidative stress, these findings confirm the effective antioxidant property of the nanovesicles by reducing intracellular MDA production.

### Example 8: Improvement of Collagen Production in Human Fibroblasts and Restoration of Oxidative Stress Damage in Myoblasts by Rocket-, Rosemary-, and Coffee-Derived Nanovesicles

Fibroblasts are specialized cells responsible for synthesizing and maintaining the extracellular matrix (ECM) of connective tissues. Collagen is the most abundant protein in the human body and plays a crucial role in providing structural support, elasticity, and strength to various tissues such as skin, bones, tendons, and cartilage. Fibroblasts actively produce and secrete collagen proteins into the ECM, where they form fibrous networks that provide mechanical support to tissues. Over time, collagen fibers undergo damage, resulting in reduced thickness and strength, which is closely associated with the skin aging phenomena (DOI: 10.1111/jocd.12450) since fibroblasts respond to various signals, including growth factors and mechanical cues, to regulate collagen synthesis in response to physiological demands such as tissue repair, remodeling, and growth.

According to literature, the process of collagen production by fibroblasts can be influenced by various factors, including oxidative stress. Oxidative stress occurs when there is an imbalance between the production of reactive oxygen species (ROS) and the body's antioxidant defenses, leading to cellular damage. Oxidative stress can negatively impact fibroblast function and collagen synthesis by disrupting signaling pathways involved in collagen production, altering gene expression related to ECM synthesis, and promoting collagen degradation.

Based on these considerations, the objective of the present research was the evaluation of the effect of RDVs, RVs and CVs of oxidative damage induced by H₂O₂ in vitro on human fibroblast cells.

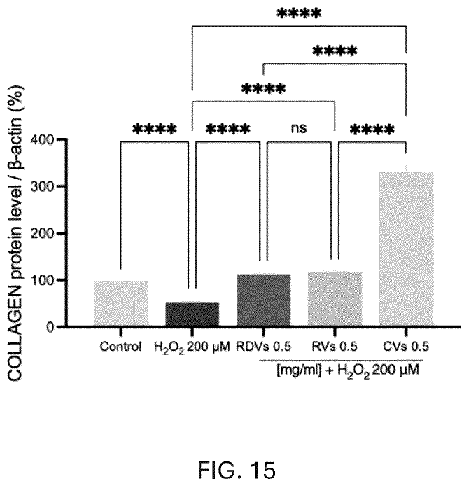

Western blotting experiments were conducted on BJ-5TA cells, where according to the experimental conditions, H₂O₂ alone induces a reduction in collagen protein synthesis up to 54.63±4.860%. Interestingly, the pretreatment with RDVs, RVs and CVs 0.5 mg/ml leads to an increase of protein levels up to 113.9±4.45% for rocket-derived nanovesicles, 118.4±2.60%. for rosemary-derived nanovesicles and 331.5±15.34% for coffee-derived nanovesicles. Results are shown in FIG. 15.

Considering this evidence, considering the strong antioxidant effects of rosemary and coffee nanovesicles, we investigated the effect of RVs and CVs 0.5 mg/ml on cellular target involved in oxidative stress (Nuclear factor erythroid 2-related factor 2, Nrf-2) and inflammatory (cyclooxygenase-2, COX2) responses, lipid metabolism (fatty acid synthase, FASN, and lipid accumulation) and glucose homeostasis (transporter protein type-4, GLUT4) in C2C12 cells.

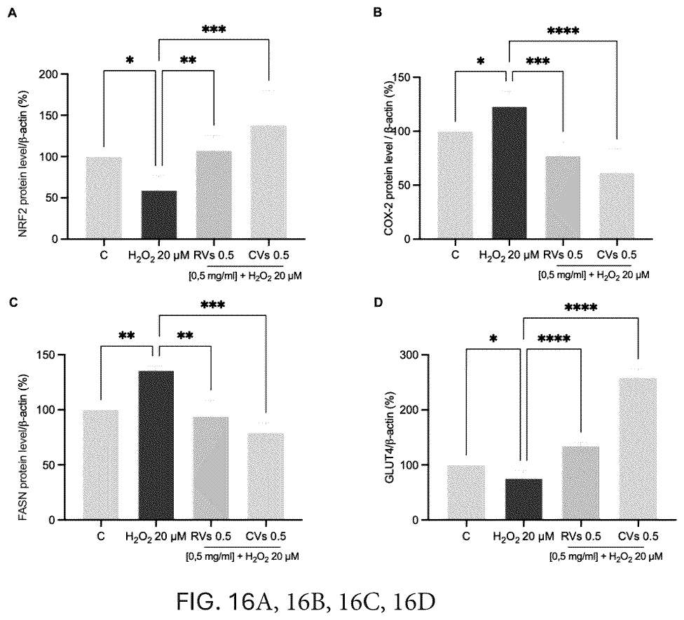

To assess the effects of RVs and CVs on the Nrf-2-pathway, western blotting experiments were performed. Our findings indicated that after the treatment of C2C12 cells with H₂O₂ (20 μM), a significant decrease of Nrf-2 protein level by 59.35±17.8% was observed versus control cells (FIG. 16A). The pretreatment with RVs and CVs produces antioxidant activity through the Nrf-2 pathway modulation in H₂O₂ treated C2C12 cells.

In fact, RVs and CVs 0.5 mg/ml increased the Nrf-2 protein levels up to 107.5±18.56% and 138.0±42.25%, respectively. FIG. 16B indicates that after the treatment of C2C12 cells with H₂O₂ (20 μM), a significant increase of COX-2 protein level by 123.2±14.48% was observed versus control cells.

Interestingly, the pretreatment with RVs and CVs 0.5 mg/ml modulates COX-2 expression in H₂O₂ treated C2C12 cells. In fact, RVs and CVs 0.5 mg/ml decreased the COX-2 protein levels up to 77.29±13.36% and 61.51±22.39%, respectively.

As shown in FIG. 16C and D, RDVs and ALVs impact on protein levels of FASN and GLUT4 in C2C12 cells. As shown in FIG. 16C, while H₂O₂ (20 μM) increases the FASN protein levels up to 135.9±4.07%, RVs and CVs 0.5 mg/ml decrease the FASN protein levels up to 94.11±14.86% and 79.13±9.47%, respectively.

Lastly, H₂O₂ (20 μM) decrease the GLUT4 protein levels up to 75.8±14.57%, RVs and CVs 0.5 mg/ml increase the GLUT4 protein levels up to 134.7±7.024% and 258.7±16.297%, respectively.

### Example 9: Identification of miRNAs from Vesicles Extracted from Rocket

To evaluate whether miRNAs enclosed in rocket-derived vesicles could contribute to the observed biological activity, we performed complete miRNA sequencing using an external service (Galseq s.r.l.). The bioinformatic analysis revealed the presence of 1736 sequences corresponding to 181 known plant miRNAs, some of which were particularly enriched (e.g. mir165, miR166). In the following list, we have grouped miRNAs into families.

| Family | |
|---|---|
| miRNA | List of identified miRNA |
| miR156 | miR156a, miR156a.1, miR156a.2, miR156a.3, miR156b, |
| | miR156b-5p, miR156c, miR156d, miR156e, miR156f, |
| | miR156g, miR156h, miR156i, miR156j, miR156l, miR156m, |
| | miR156n, miR156o |
| miR157 | miR157a, miR157a-5p, miR157b, miR157b.1, miR157b.2, |
| | miR157b.3, miR157b.4, miR157c, miR157d |
| miR158 | miR158a, miR158b |
| miR159 | miR159a, miR159b, miR159c, miR159d, miR159j, miR159k, |
| | miR159m, miR162, miR162b |
| miR165 | miR165a, miR165b |
| miR166 | miR166a, miR166a, miR166b, miR166b.1, miR166c, miR166c, |
| | miR166c.1, miR166c.2, miR166c.3, miR166c.4, miR166d, |
| | miR166d.1, miR166d.2, miR166d.3, miR166e, miR166f, |
| | miR166g, miR166h, miR166i, miR166j, miR166k, miR166l, |
| | miR166m, miR166n, miR166o, miR166p, miR166q, miR166r, |
| | miR166s, miR166t |
| miR167 | miR167a, miR167b, miR167c, miR167d, miR167e, miR167f, |
| | miR167g, miR167h, miR167i, miR167j, miR167k, miR167l, |
| | miR167m, miR167n, miR167o, miR167p, miR167q, miR167r, |
| | miR167s, miR167t |
| miR319 | miR319a, miR319a.1, miR319a.2, miR319b, miR319c, |
| | miR319d, miR319e, miR319f, miR319g, miR319h |
| miR390 | miR390a, miR390a.1, miR390a.3, miR390a-5p, miR390b, |
| | miR390c, miR390d |
| miR394 | miR394a, miR394a.1, miR394a.2, miR394a.3, miR394a.4, |
| | miR394a-5p, miR394b, miR394b-5p |
| miR403 | miR403a, miR403b, miR403c, miR403d, miR403e, miR403f |
| miR408 | miR408.1, miR408.2, miR408-5p, miR408b |
| Altri | miR168, miR2005, miR2910, miR2911, miR2914, miR395c, |
| | miR396b, miR398, miR4995, miR5368, miR59-npr, |
| | miRf10192-akr, miRf10258-akr, miRf10482-akr, miRf10804- |
| | akr, miRf12412-akr, miRf12524-akr, miR58-1-npr, miR58-2- |
| | npr, miR58-3-npr, miR21-1-npr, miR21-2-npr |

### Example 10: Stability of the Nanovesicles According to the Present Invention

The vesicles obtained from rocket according to the present invention protocol were spray dried and then stored for up to two years at room temperature. Stability analysis were performed and results are reported in table 8 below. The samples are stable up to two year.

TABLE 8

| Time (months) | D10 (nm) | D50 (nm) | D90 (nm) | Particles/ml |
|---|---|---|---|---|
| 0 | 109 ± 2 | 154 ± 3 | 278 ± 5 | 7.8 (±0.1) * 10⁸ |
| 6 | 112 ± 1 | 155 ± 4 | 265 ± 12 | 5.3 (±0.1) * 10⁸ |
| 24 | 99.5 ± 6.3 | 138 ± 11.3 | 232 ± 21.2 | 5.3 (±0.1) * 10⁸ |

### Example 11: Comparison of Different Extraction Methods

Starting from the same starting material, i.e. rocket (Eruca sativa) leaves, three different extraction protocols have been applied in parallel, as follows:

### Protocol 1, According to the Invention.

The matrix (200 g) has been washed and macerated overnight at +4° C. in Tris-HCl.

The resulting homogenate was blended at 200 Watt, 1′. After sieving, the following centrifugation steps were performed:

$$700 g \times {10}^{′} at 4 ° C .$$

$$700 g \times {10}^{′} at 4 ° C .$$

$$1500 g \times {20}^{′} at 4 ° C .$$

At the end, the supernatant was collected and centrifuge at 3000 g×30′ at 4° C.
Cut off 100 at 4000 g×30′ at 4° C.
Cut off 10 4000 g×10′ at 4° C. . . .
SEC 35 nm eluted in Tris-HCL

### Protocol 2, Comparative.

The matrix (200 g) has been washed and cut into pieces, then passed into a juicer. The resulting homogenate was filtered through a cotton cloth and then ultracentrifugated as follows:
8,000×g for 1 hour at 4° C.→collect supernatant
20,000×g for 1 hour at 4° C.→collect supernatants
Store at 80° C.
Cut off 10→13 ml, 5,000×g for 3 hours at 4° C.
SEC qEV original/70 nm eluted in PBS

### Protocol 3, Comparative.

The matrix (200 g) has been washed and cut into pieces, then passed into a juicer. The resulting homogenate was extracted with a commercially available extractor. Juices were then centrifuged at 500×g for 10 min at 10° C. The supernatants were filtered with 100 μm filters and serially centrifugated at 2000×g for 20 min and then at 15,000×g for 30 min at 10° C. The supernatants were subsequently ultracentrifuged in a Sorvall WX Ultracentrifuge Series (Thermo Fisher Scientific, Waltham, MA, USA) at 110,000×g for 1 h 30 min at 10° C. to collect the so called Exocomplex® (Exolab, Italia). The pellet was resuspended in ultra-filtered and sterilized water (or in PBS) for downstream analyses and preserved at +4° C.

The obtained samples were evaluated by Dynamic Light Dispersion (DLS) and Nanoparticle Tracking Analysis (NTA), following the methodology described in the method section above. The results are the following:

### Protocol 1, According to the Invention

The Cryo EM images (FIG. 17A, B) confirm the presence of microvesicles into the sample.

### Protocol 2 (Comparative)

The sample does not contain plant vesicles. Exemplificative picture taken with Cryo-EM is reported in FIG. 18.

### Protocol 3 (Comparative)

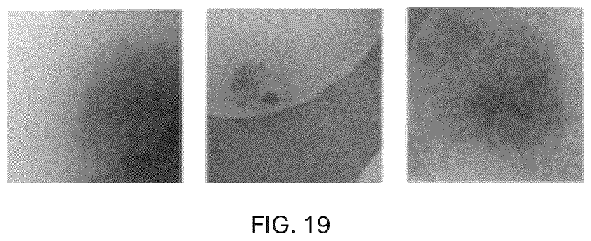

The sample does not contain plant vesicles. Exemplificative picture taken with Cryo-EM is reported in FIGS. 19A, B, C. A good protein extraction is obtained but very few vesicles are comprised.

### Example 12: Extraction of Vesicles from Banana Pulp

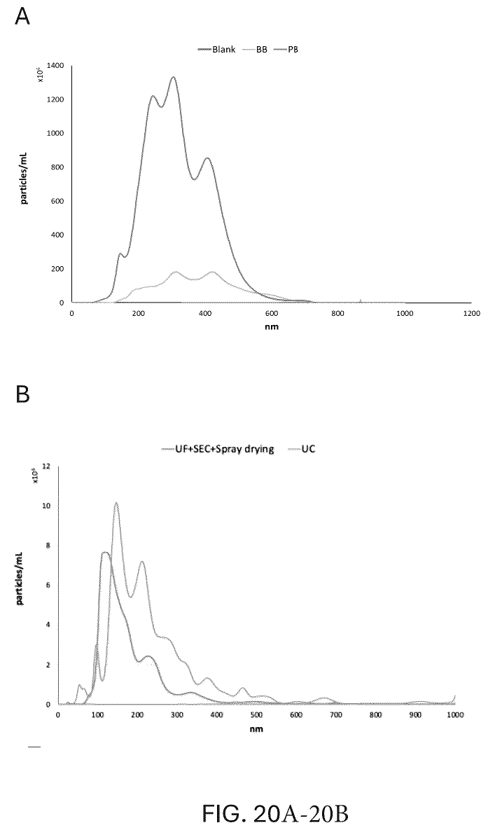

The method according to the present invention does not allow plant vesicles obtaining from banana pulp and peel, as demonstrated by NTA results (FIG. 20A). This shows the specificity of the method on selected plant varieties. In parallel, the experiment was performed on rocket (FIG. 20B), confirming the validity of the protocol according to the present invention.

### Example 13: In-Vivo Antiaging Activity Exerted by Rosemary-Derived Nanovesicles

Experimental protocol: a panel composed of 20 females aged 39-65 years was involved in the experimentation. The duration of the study was 15 days, during which participants applied twice a day, morning and evening, on the area of interest the composition according to the invention.

The tested composition consists of: 30 ml di rosemary derived nanovesicles (3.23E+10/mL) in Tris-HCl+570 mL H₂O.

Instrumental analyses were performed ad day 0 and at day 15.

The instrumental analyses were:

### Corneometer CM 825

Assessment of stratum corneum hydration on an arbitrary scale (0-100 U.A.). A probe with a surface area of 49 mm² is used to obtain measurements of 10-20 μm depth in the stratum corneum.

### Cutometer Dual MPA580

Assessment of the viscoelastic properties of the skin using an instrument that measures the vertical deformation of the skin induced by suction, expressed in mm. A negative pressure of 450 mbar is applied to the skin using a probe with a diameter of 2 mm for a period of 1 to 3 seconds. Each suction is followed by a release time that allows the skin to return to its resting condition.

Parameter investigated: R0: Maximum Extension (mm). A decrease in maximum extension determines an increase in skin firmness.

### Antera 3D

Acquisition of a high-resolution image of the skin surface using an optical system and mathematical algorithms to obtain three-dimensionality and measurement, using special software, of skin topographical parameters.

### Parameter Investigated: Ra—Average Roughness (μm).

The study surprisingly demonstrated increased skin hydration (+3.26% in 15 days); improved skin firmness (+4.27% in 15 days); improved skin texture decreasing the mean roughness (−4.26% in 15 days).

A tolerability test confirmed no adverse skin reactions are elicited with the composition.

1. A method for the production, purification and stabilization of plant-derived nanovesicles (PDVs), comprising the following steps:
a. Homogenization and sieving of a plant matrix to obtain a homogenate;
b. Separation of the juice from the solid residue present in the homogenate obtained in step a);
c. Isolation and purification of the PDVs contained in the extract thus obtained via ultrafiltration, Tangential Flow Filtration (TFF), and/or size exclusion chromatography (SEC);
d. Stabilization of said PDVs by spray-drying.

2. The method according to claim 1, characterized by the fact that said plant matrix in step a) is a plant matrix from a vegetable having a low sugar content, preferably said vegetable is rocket, rosemary, coffee.

3. The method according to claim 1, characterized in that said homogenate in step a) has a viscosity comprised between 1.5 mPa·s and 9.5 mPa·s, preferably between 2 mPa·s and 9 mPa·s.

4. The method according to claim 1, characterized in that said juice is separated in step b) by differential centrifugation, and, optionally, by TFF.

5. The method according to claim 1, characterized by the fact that said PDVs in step c) are isolated with the aid of one or more ultrafiltering membranes.

6. The method according to claim 5, characterized by the fact that said PDVs in step c) are isolated with the aid of two ultrafiltering membranes having molecular cutoffs of 100 kDa and 10 kDa.

7. The method according to claim 1, wherein TFF has been performed and said PDVs are isolated with the aid of a single ultrafiltering membrane having molecular cutoffs of 10 kDa.

8. The method according to claim 1, characterized in that said purification in step c) occurs with size exclusion chromatography.

9. PDVs obtained by the method according to claim 1.

10. The PDVs according to claim 9, comprising nanovesicles and lipid particles, said nanovesicles having an average particle diameter comprised between 80 nm and 280 nm, preferably between 120 and 180 nm.

11. A pharmaceutical, nutraceutical, or cosmetic formulation comprising the PDVs according to claim 9 and at least one physiologically acceptable excipient.

12. PDVs according to claim 8 for hypocholesterolemic and/or hypolipidemic and/or hypoglycemic use.

13. PDVs according to claim 8 for use as anti-ageing and antioxidant.
